(19) United States
(12) Patent Application Publication
(10) Pub. No.: US 2017/0143622 A1
(43) Pub. Date: May 25, 2017

(54) STABLE LIQUID READY-TO-USE INJECTABLE FORMULATION OF BORTEZOMIB

(71) Applicant: DR. REDDY'S LABORATORIES LIMITED, Hyderabad (IN)

(72) Inventors: Riyaz Ahammad SHAIK, Hyderabad (IN); Akash CHAURASIYA, Agra (IN); Ananya SAHA, Asansol (IN); Bhavesh Vallabhbhai PATEL, Hyderabad (IN); Harshal BHAGWATWAR, Hyderabad (IN); Sumitra Ashok PILLAI, Hyderabad (IN)

(21) Appl. No.: 15/323,488

(22) PCT Filed: Jul. 6, 2015
(86) PCT No.: PCT/IB2015/055092

§ 371 Date: Jan. 3, 2017

(30) Foreign Application Priority Data
Jul. 4, 2014 (IN) 3322/CHE/2014

(51) Int. Cl. A61K 9/00; A61K 47/10; A61K 47/16; A61K 31/69

(52) U.S. Cl. CPC A61K 9/0019; A61K 31/69; A61K 47/10; A61K 47/16

(57) ABSTRACT

Aspects of the present invention provide stable liquid ready-to-use injectable formulation of bortezomib or pharmaceutically acceptable salts thereof. Another aspect of the present invention provides processes for preparation of such stable liquid ready-to-use injectable formulation of bortezomib and methods of using such formulations for treating various types of cancers in mammals.

### FIELD OF INVENTION

Aspects of the present invention provide stable liquid ready-to-use injectable formulation of bortezomib or pharmaceutically acceptable salts thereof. Another aspect of the present invention provides processes for preparation of such stable liquid ready-to-use injectable formulation of bortezomib and methods of using such formulations for treating various types of cancers.

### BACKGROUND OF THE INVENTION

Bortezomib [(1R)-3-methyl-1-[[(2S)-1-oxo-3-phenyl-2[(pyrazinylcarbonyl)amino]propyl]amino]butyl]boronic acid is 26S proteasome inhibitor and used as anti-neoplastic agent for the treatment of relapsed multiple myeloma and mantle cell lymphoma. Bortezomib is structurally represented as

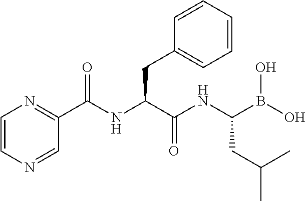 [SMILES: CC(C)C[C@H](NC(=O)[C@H](Cc1ccccc1)NC(=O)c1cnccn1)B(O)O]

U.S. Pat. No. 5,780,454 describe the Bortezomib, related compounds and synthetic process of preparation thereof.

Butyl boronic acids (includes Bortezomib) are readily oxidized by air to generate 1-butanol and boric acid, which limit the pharmaceutical utility of boronic acid compounds, complicating the characterization of pharmaceutical agents that comprise the boronic acid compounds and limit their shelf-life.

U.S. Pat. Nos. 6,958,319, 6,713,446, 6,297,217 & 7119080 discloses formation of di-ester of boronic acid functional group in bortezomib and the like, with mannitol after lyophilization. To circumvent the issues with stability of bortezomib in water and well known non-aqueous solvents, bortezomib is lyophilized and reconstituted prior to injection. Commercially, bortezomib is sold as mannitol ester under the brand name VELCADE® marketed by Millennium Pharma, which is supplied as a sterile lyophilized powder for intravenous infusion in single-dose vials. As per the label of VELCADE® approved by USFDA, each single dose vial contains 3.5 mg of bortezomib and 35 mg mannitol and should be reconstituted with 0.9% sodium chloride to a final concentration of 2.5 mg/ml or 1 mg/ml of bortezomib before administration. VELCADE®, when reconstituted, forms a solution consisting of the mannitol ester of bortezomib in equilibrium with free bortezomib. As instructed in the USFDA approved label, the reconstituted material may be stored in the original vial and/or the syringe prior to administration. Such reconstituted product may be stored for up to 8 hours in a syringe; however, total storage time must not exceed 8 hours when exposed to normal indoor lighting.

International application publication No. WO 2010/089768 discloses the approaches to stabilize bortezomib that include lyophilized formulations of bortezomib with tromethamine. However, all these formulations failed to provide storage stable liquid formulations having sufficient stability for commercially relevant period of time, especially which are not obtained by reconstitution of a lyophilized product.

Though lyophilization approach solves the issues associated with stability of bortezomib, lyophilization involves complex manufacturing processes, which in turn results in increasing manufacturing costs. Before administration to patients in clinics or hospitals, such lyophilized powder formulation must be reconstituted with suitable fluids to achieve desired concentration for administration. Therefore, an additional step of reconstitution is mandatory for such lyophilized formulation which causes inconveniences raising safety issues and risks of contamination by microorganisms. In addition, improper reconstitution may lead to high or low dosing to the patient in need. Moreover, the reconstituted solutions of bortezomib are not suitable for administration for up to only 8 hours when stored at room temperature.

On the other hand, development of stable liquid ready-to-use injectable formulation of bortezomib was also found challenging in light of the prior arts.

Wu et al. in “Degradation pathways of a peptide boronic acid derivative, 2-Pyz-(CO)-Phe-Leu-B(OH)(2)” Journal of Pharmaceutical Sciences, 2000, 89(6): 758-65, discloses initial pre-formulation study results of bortezomib and possible degradation products in various conditions. Wu reports that when in a polyethylene glycol (PEG) 300: Ethanol:Water (40:10:50) solvent system, significant (as much as 20%) quantities of degradation was observed at 25° C. in 24 hours. Further, in ethanol: normal saline solution (2:98, pH 2.8), bortezomib (0.5 mg/ml) degraded 20% at 25° C. in 1 month. In propylene glycol (PG): Ethanol:Water (50:10:40), the stability of bortezomib was improved but still degraded 20% in 8 months when stored at 25° C. As a whole Wu article teaches that bortezomib shows erratic stability behavior while formulated as liquid solutions containing water and well known non-aqueous solvents frequently used in parenteral dosage forms.

International application publication No. WO 2010/039762 discloses sugar free physical admixtures, lyophilized preparations or ready-to-use solutions, comprising bortezomib. The specification of WO'762 publication does not disclose stability data for any liquid ready-to-use solutions of bortezomib proven stability.

U.S. Pat. No. 8,263,578 discloses a storage-stable liquid injectable composition that includes Bortezomib, wherein the composition comprise a single-phase liquid formulation comprising a ‘substantially non-aqueous solvent system’ suitable for injection wherein the solvent system comprises propylene glycol as a predominant component. As per the definition provided in the specification of '578 patent, ‘substantially non-aqueous solvent system’ refers to a solvent system in which bortezomib is completely soluble without water and that comprises water in a total amount of equal or less that 15% v/v. '578 patent teaches that propylene glycol (PG) provides storage stable liquid injectable composition of bortezomib, wherein in other non-aqueous solvents bortezomib encountered stability or solubility issues. As reported in table 2 of '578 bortezomib degraded to 76% in ethanol composition while stored at 40° C. and 75% relative humidity for 1 month. Another non-aqueous solvent, polyethylene glycol (PEG) was not included in the study due to insolubility of bortezomib in PEG. Further, only high amount of PG was found suitable for storage-stable liquid bortezomib composition. Such liquid compositions with high amount of PG as non-aqueous solvent, was found to have unacceptable osmolality, which in turn can cause significant toxicity when administered directly without further dilution by subcutaneous and intravenous routes.

Surprisingly, inventors of the present application discovered liquid ready-to-use injectable formulation of bortezomib comprising solvent system comprising water as predominant component, which are stable for commercially significant time and also suitable for administration by subcutaneous and intravenous routes without further dilution.

### SUMMARY OF INVENTION

The invention provides a stable liquid ready-to-use injectable formulation of bortezomib or pharmaceutically acceptable salts thereof comprising solvent system suitable for injection, wherein the solvent system comprises less than about 50% v/v of non-aqueous solvents.

In embodiments, the stable liquid ready-to-use injectable formulation comprises from about 0.01 mg/ml to about 25 mg/ml or from about 0.1 mg/ml to about 15 mg/ml or from about 0.5 mg/ml to about 5 mg/ml of bortezomib or pharmaceutically acceptable salts thereof.

In one embodiment, the solvent system comprises mixture of aqueous and non-aqueous solvents.

In embodiments, the solvent system comprises less than about 45% v/v or about 35% v/v, or about 25% v/v or about 15% v/v or about 10% v/v or about 5% v/v of non-aqueous solvents.

In one embodiment, the solvent system comprises from about 0.5% v/v to about 20% v/v or 2% v/v to about 15% v/v or from about 5% v/v to about 10% w/v or from about 1% v/v to about 8% v/v of non-aqueous solvents.

In another embodiment, the solvent system is free of non-aqueous solvent.

In embodiments, the non-aqueous solvent is selected from the group of ethanol, methanol, acetone, acetonitrile, 1-Butanol, 2-butanol, tertiary butanol, isopropanol, 1-propanol, dimethyl sulfoxide and dimethylacetamide.

In another embodiment, the solvent system comprises about 100% v/v of water.

In embodiments, the stable liquid ready-to-use injectable formulation of bortezomib or pharmaceutically acceptable salts thereof comprise solvent system suitable for injection and at least one pharmaceutically acceptable excipient selected form the group of solubilizer, stabilizer, buffering agent, tonicity contributing agent, pH adjusting agent, wherein the solvent system comprises less than about 50% v/v of non-aqueous solvents.

The invention also provides a stable liquid ready-to-use injectable formulation comprising bortezomib, or pharmaceutically acceptable salts; and a solvent system suitable for injection, wherein the solvent system comprises the mixture of aqueous and non-aqueous solvents and wherein the solvent system comprises less than about 50% v/v of non-aqueous solvents.

The invention further provides a stable liquid ready-to-use injectable formulation comprising bortezomib, or pharmaceutically acceptable salts; and solvent system suitable for injection, wherein the solvent system comprises the mixture of aqueous and non-aqueous solvents and wherein the solvent system comprises from about 5% v/v to about 15% v/v of non-aqueous solvents.

The invention provides a stable liquid ready-to-use injectable formulation comprising bortezomib, or pharmaceutically acceptable salts thereof; and solvent system suitable for injection, wherein the solvent system comprises the mixture of water and ethanol.

The invention also provides a stable liquid ready-to-use injectable formulation comprising bortezomib, or pharmaceutically acceptable salts thereof; and solvent system suitable for injection, wherein the solvent system comprises the mixture of water and ethanol, and wherein the solvent system comprises less than about 50% v/v of ethanol.

The invention further provides a stable liquid ready-to-use injectable formulation comprising bortezomib, or pharmaceutically acceptable salts thereof; and solvent system suitable for injection, wherein the solvent system comprises the mixture of water and ethanol and wherein the solvent system comprises from about 5% v/v to about 15% v/v of ethanol.

The invention further provides a stable liquid ready-to-use injectable formulation comprising bortezomib, or pharmaceutically acceptable salts thereof; solubilizer, and a solvent system.

In embodiments, the solubilizer is selected from the group of carboxylic acid or derivative thereof, tromethamine and sugar.

The invention also provides a stable liquid ready-to-use injectable formulation comprising bortezomib, or pharmaceutically acceptable salts thereof; solubilizer and a solvent system suitable for injection, wherein the solvent system comprises the mixture of aqueous and non-aqueous solvents and wherein the solvent system comprises less than about 50% v/v of non-aqueous solvents.

The invention further provides a stable liquid ready-to-use injectable formulation comprising bortezomib, or pharmaceutically acceptable salts thereof; about 0.01 mg/mL to about 50 mg/mL of solubilizer and a solvent system suitable for injection, wherein the solvent system comprises the mixture of aqueous and non-aqueous solvents and wherein the solvent system comprises from about 5% v/v to about 15% v/v of non-aqueous solvents.

The invention further provides a stable liquid ready-to-use injectable formulation comprising bortezomib, or pharmaceutically acceptable salts thereof; citric acid, and a solvent system.

The invention also provides a stable liquid ready-to-use injectable formulation comprising bortezomib, or pharmaceutically acceptable salts; citric acid and a solvent system suitable for injection, wherein the solvent system comprises the mixture of water and ethanol and wherein the solvent system comprises less than about 50% v/v of ethanol.

The invention also provides a stable liquid ready-to-use injectable formulation comprising bortezomib, or pharmaceutically acceptable salts thereof; citric acid and a solvent system suitable for injection, wherein the solvent system comprises the mixture of water and ethanol and wherein the solvent system comprises from about 5% v/v to about 15% v/v of ethanol.

The invention further provides a stable liquid ready-to-use injectable formulation comprising bortezomib, or pharmaceutically acceptable salts thereof; about 0.01 mg/mL to about 10 mg/mL of citric acid and a solvent system suitable for injection, wherein the solvent system comprises the mixture of water and ethanol and wherein the solvent system comprises from about 5% v/v to about 15% v/v of ethanol.

The invention further provides a stable liquid ready-to-use injectable formulation comprising bortezomib, or pharmaceutically acceptable salts thereof; about 2.5 mg/mL of citric acid and a solvent system suitable for injection, wherein the solvent system comprises the mixture of water and ethanol and wherein the solvent system comprises from about 5% v/v to about 15% v/v of ethanol.

The invention also provides a stable liquid ready-to-use injectable formulation comprising bortezomib, or pharmaceutically acceptable salts thereof; solubilizer, buffering agent and a solvent system.

In embodiments, the buffering agent is selected from the group of sodium acetate, acetic acid, sodium citrate, citric acid, tromethamine and phosphate.

The invention also provides a stable liquid ready-to-use injectable formulation comprising bortezomib, or pharmaceutically acceptable salts thereof; solubilizer, buffering agent and a solvent system suitable for injection, wherein the solvent system comprises the mixture of aqueous and non-aqueous solvents and wherein the solvent system comprises less than about 50% v/v of non-solvents.

The invention further provides a stable liquid ready-to-use injectable formulation comprising bortezomib, or pharmaceutically acceptable salts thereof; about 0.01 mg/mL to about 50 mg/mL of solubilizer, about 0.01 mg/mL to about 10 mg/mL of buffering agent and a solvent system suitable for injection, wherein the solvent system comprises the mixture of aqueous and non-aqueous solvents and wherein the solvent system comprises from about 5% v/v to about 15% v/v of non-aqueous solvents.

The invention further provides a stable liquid ready-to-use injectable formulation comprising bortezomib, or pharmaceutically acceptable salts thereof; citric acid, sodium citrate and a solvent system.

The invention also provides a stable liquid ready-to-use injectable formulation comprising bortezomib, or pharmaceutically acceptable salts thereof; citric acid, sodium citrate and a solvent system suitable for injection, wherein the solvent system comprises the mixture of water and ethanol and wherein the solvent system comprises less than about 50% v/v of ethanol. The invention also provides a stable liquid ready-to-use injectable formulation comprising bortezomib, or pharmaceutically acceptable salts thereof; citric acid, sodium citrate and a solvent system suitable for injection, wherein the solvent system comprises the mixture of water and ethanol and wherein the solvent system comprises from about 5% v/v to about 15% v/v of ethanol.

The invention further provides a stable liquid ready-to-use injectable formulation comprising bortezomib, or pharmaceutically acceptable salts thereof; about 0.01 mg/mL to about 10 mg/mL of citric acid, about 0.01 mg/mL to about 10 mg/mL of sodium citrate, and a solvent system suitable for injection, wherein the solvent system comprises the mixture of water and ethanol and wherein the solvent system comprises from about 5% v/v to about 15% v/v of ethanol, and wherein the composition has a pH of 3 to 7.

The invention further provides a stable liquid ready-to-use injectable formulation comprising bortezomib, or pharmaceutically acceptable salts thereof; about 2.5 mg/mL of citric acid, about 6 mg/mL of sodium citrate and a solvent system suitable for injection, wherein the solvent system comprises the mixture of water and ethanol and wherein the solvent system comprises from about 5% v/v to about 15% v/v of ethanol.

The invention further provides a stable liquid ready-to-use injectable formulation comprising bortezomib, or pharmaceutically acceptable salts thereof; about 2.5 mg/mL of citric acid, about 6 mg/mL of sodium citrate and a solvent system suitable for injection, wherein the solvent system comprises the mixture of water and ethanol and wherein the solvent system comprises from about 5% v/v to about 15% v/v of ethanol and wherein the composition has a pH of about 3 to 7.

The invention also provides a stable liquid ready-to-use injectable formulation comprising bortezomib, or pharmaceutically acceptable salts thereof; solubilizer, buffering agent, stabilizer and a solvent system.

In embodiments, the stabilizer is selected from the group of sodium metabisulfite, sodium sulphite, sodium bisulfate, sodium thiosulfate, TPGS (d-alpha tocopheryl polyethylene glycol 1000 succinate), sodium acetate trihydrate, sodium formaldehyde sulfoxylate, tocopherol, ascorbic acid, EDTA and derivatives, monothioglycerol, methionine, thiourea, cysteine, butylated hydroxy toluene (BHT), butylated hydroxy anisole (BHA) and propyl gallate.

The invention also provides a stable liquid ready-to-use injectable formulation comprising bortezomib, or pharmaceutically acceptable salts thereof; solubilizer, buffering agent, stabilizer and a solvent system suitable for injection, wherein the solvent system comprises the mixture of aqueous and non-aqueous solvents and wherein the solvent system comprises less than about 50% v/v of non-aqueous solvents.

The invention also provides a stable liquid ready-to-use injectable formulation comprising bortezomib, or pharmaceutically acceptable salts thereof; solubilizer, buffering agent, stabilizer and a solvent system suitable for injection, wherein the solvent system comprises the mixture of aqueous and non-aqueous solvents and wherein the solvent system comprises from about 5% v/v to about 15% v/v of non-aqueous solvents.

The invention further provides a stable liquid ready-to-use injectable formulation comprising bortezomib, or pharmaceutically acceptable salts thereof; about 0.01 mg/mL to about 10 mg/mL of solubilizer, about 0.01 mg/mL to about 10 mg/mL of buffering agent, about 0.5 mg/mL of stabilizer and a solvent system suitable for injection, wherein the solvent system comprises the mixture of aqueous and non-aqueous solvents and wherein the solvent system comprises from about 5% v/v to about 15% v/v of non-aqueous solvents.

The invention further provides a stable liquid ready-to-use injectable formulation comprising bortezomib, or pharmaceutically acceptable salts thereof; about 0.01 mg/mL to about 10 mg/mL of solubilizer, about 0.01 mg/mL to about 10 mg/mL of buffering agent, about 0.5 mg/mL of stabilizer and a solvent system suitable for injection, wherein the solvent system comprises the mixture of aqueous and non-aqueous solvents and wherein the solvent system comprises from about 5% v/v to about 15% v/v of non-aqueous solvent, and wherein the composition is from about wherein the composition has a pH of about 3 to about 7.

The invention further provides a stable liquid ready-to-use injectable formulation comprising bortezomib, or pharmaceutically acceptable salts thereof; citric acid, sodium citrate, EDTA and a solvent system.

The invention also provides a stable liquid ready-to-use injectable formulation comprising bortezomib, or pharmaceutically acceptable salts thereof; citric acid, sodium citrate, EDTA and a solvent system suitable for injection, wherein the solvent system comprises the mixture of water and ethanol and wherein the solvent system comprises less than about 50% v/v of ethanol.

The invention also provides a stable liquid ready-to-use injectable formulation comprising bortezomib, or pharmaceutically acceptable salts thereof; citric acid, sodium citrate, EDTA and a solvent system suitable for injection, wherein the solvent system comprises the mixture of water and ethanol and wherein the solvent system comprises from about 5% v/v to about 15% v/v of ethanol.

The invention further provides a stable liquid ready-to-use injectable formulation comprising bortezomib, or pharmaceutically acceptable salts thereof; about 0.01 mg/mL to about 10 mg/mL of citric acid, about 0.01 mg/mL to about 10 mg/mL of sodium citrate, about 0.5 mg/mL of EDTA and a solvent system suitable for injection, wherein the solvent system comprises the mixture of water and ethanol and wherein the solvent system comprises from about 5% v/v to about 15% v/v of ethanol.

The invention further provides a stable liquid ready-to-use injectable formulation comprising bortezomib, or pharmaceutically acceptable salts thereof; about 2.5 mg/mL of citric acid, about 6 mg/mL of sodium citrate and about 0.5 mg/mL of EDTA and a solvent system suitable for injection, wherein the solvent system comprises the mixture of water and ethanol and wherein the solvent system comprises from about 5% v/v to about 15% v/v of ethanol.

The invention further provides a stable liquid ready-to-use injectable formulation comprising bortezomib, or pharmaceutically acceptable salts thereof; about 2.5 mg/mL of citric acid, about 6 mg/mL of sodium citrate and about 0.5 mg/mL of EDTA and a solvent system suitable for injection, wherein the solvent system comprises the mixture of water and ethanol and wherein the solvent system comprises from about 5% v/v to about 15% v/v of ethanol and wherein the composition has a pH of about 3 to 7.

The invention also provides a stable liquid ready-to-use injectable formulation of bortezomib or pharmaceutically acceptable salts thereof comprising solvent system suitable for injection comprising less than about 50% v/v of non-aqueous solvents, wherein the formulation comprises not more than about 10% of total impurities.

In embodiments, the formulation comprises not more than about 10% of total impurities and not more than about 5% of single maximum individual impurity.

In embodiments, the formulation comprises not more than about 8% of total impurities and not more than about 4% of single maximum individual impurity.

### DETAILED DESCRIPTION

The invention provides a stable liquid ready-to-use injectable formulation of bortezomib or pharmaceutically acceptable salts thereof comprising solvent system suitable for injection, wherein the solvent system comprises less than about 50% v/v of non-aqueous solvents.

In embodiments, the stable liquid ready-to-use injectable formulation comprises from about 0.01 mg/ml to about 25 mg/ml or from about 0.1 mg/ml to about 15 mg/ml or from about 0.5 mg/ml to about 10 mg/ml of bortezomib or pharmaceutically acceptable salts thereof. More preferably the formulation of this invention comprise from about 1 mg/mL to about 5 mg/mL of bortezomib or pharmaceutically acceptable salts thereof; and most preferably it contains 1 mg/mL and 2.5 mg/mL of bortezomib or pharmaceutically acceptable salts thereof. In one preferred embodiment, the concentration of bortezomib is 1 mg/ml. In another preferred embodiment, the concentration of bortezomib is 2.5 mg/ml.

The term “bortezomib” includes the compound bortezomib, pharmaceutically acceptable salts thereof, isomers, solvates, prodrugs, complexes and hydrates, anhydrous forms thereof, and any polymorphic or amorphous forms or combinations thereof.

As used herein, the term “pharmaceutically acceptable salts” includes bortezomib salts with bases, such as, those formed from the alkali metals, alkaline earth metals, non-toxic metals, and mono-, di- and trisubstituted amines, for example the sodium, potassium, lithium, calcium, magnesium, aluminum, zinc, ammonium, trimethylammonium, triethanolammonium, pyridinium, and substituted pyridinium salts.

As used herein, the term “liquid ready-to-use” refers to a liquid for parenteral administration that is not obtained by reconstituting a lyophilized product. The liquid ready-to-use solutions as per this invention are not diluted with any diluent before administration.

As used herein, terms “composition” and “formulation” refer to preparations comprising bortezomib or pharmaceutically acceptable salts thereof; in a form suitable for administration to a mammal.

As used herein, the term “stable formulation” refers to any preparation of bortezomib or pharmaceutically acceptable salts thereof having sufficient physical and chemical stability to allow storage at a convenient temperature, such as between about 0° C. and about 50° C., for a commercially reasonable period of time. The term “physical stability” refers to maintenance of color, dissolved oxygen level, head space oxygen level, and particulate matter and the term “chemical stability” relates to formation of drug-related impurities in terms of total impurity, single maximum individual impurity and maximum individual unknown impurity. For the purpose of the present invention chemical stability also includes maintenance of pH of the finished formulation. For pharmaceutical products, stability is required for commercially relevant times after manufacturing, such as for about 6, 12, 18, 24 or 36 months, during which a product is kept in its original packaging under specified storage condition.

As used herein, the term “solvent” refers to an ingredient used for dissolving an ingredient, which is suitable for parenteral administration.

As used herein, the term “predominant solvent” or “substantial solvent” refers to a solvent in which bortezomib is completely soluble and that comprise total amount less than or equal to 40% v/v of the total solvent system.

As used herein, the term ‘less than specific concentration’ also includes ‘0%’.

Suitable solvents comprise aqueous solvent, non-aqueous solvent or suitable mixture thereof.

Suitable non-aqueous solvents comprise, but not limited to, polar protic solvents and polar aprotic solvents or mixtures thereof. The polar protic solvents are known in the art and include alkyl alcohols, for example ethanol, methanol, 1-butanol, 2-butanol, 1-propanol, isopropanol, tertiary butanol; acetic acid, alkyl glycols for example, ethylene glycol, propylene glycol and butylene glycol; glycerin; polysorbates for examples TWEEN 20, TWEEN 40 and TWEEN 80; and cyclodextrins (such as hydroxypropyl-β-cyclodextrin); polyalkylene glycols, such as polyethylene glycol, polypropylene glycol, and polybutylene glycol, acetone, acetonitrile or mixtures thereof. The polar aprotic solvents are known in the art and include dimethyl acetamide, dimethyl sulfoxide, acetone, tetrahydrofuran, 1,4-dioxone, acetonitrile, dimethyl formamide, propylene carbonate, chloroform, dichloromethane, ethyl ether, 1-methyl-2-pyrrolidione, 1,3-dimethyl-2-imidazolidinone or mixtures thereof.

In embodiments, suitable non-aqueous solvent is selected from the group of ethanol, methanol, acetone, acetonitrile, 1-Butanol, 2-butanol, tertiary butanol, isopropanol, 1-propanol, dimethyl sulfoxide and dimethylacetamide.

In embodiments, the solvent system comprises about 45% v/v or about 40% or about 35% v/v, or about 30% v/v or about 25% v/v or about 20% v/v or about 15% v/v or about 14% v/v or about 13% v/v or about 12% v/v or about 11% v/v or about 10% v/v or about 9% v/v or about 8% v/v or about 7% v/v or about 6% v/v or about 5% v/v or about 4% v/v or about 3% v/v or about 2% v/v or about 1% v/v or about 0.5% v/v of non-aqueous solvents.

In embodiments, the solvent system comprises from about 0.5% v/v to about 20% v/v or 2% v/v to about 15% v/v or from about 5% v/v to about 10% w/v or from about 1% v/v to about 8% v/v of non-aqueous solvents.

In another embodiment, the solvent system comprises about 100% v/v of water.

In embodiments, the stable liquid ready-to-use injectable formulation of bortezomib or pharmaceutically acceptable salts thereof comprise solvent system suitable for injection and at least one pharmaceutically acceptable excipient selected form the group of solubilizer, stabilizer, buffering agent, tonicity contributing agent, pH adjusting agent, wherein the solvent system comprises less than about 50% v/v of non-aqueous solvents.

The invention also provides a stable liquid ready-to-use injectable formulation comprising bortezomib, or pharmaceutically acceptable salts; and a solvent system suitable for injection, wherein the solvent system comprises the mixture of aqueous and non-aqueous solvents and wherein the solvent system comprises less than about 50% v/v of non-aqueous solvents.

A further embodiment of the invention relates to a stable liquid ready-to-use injectable formulation comprising bortezomib, or pharmaceutically acceptable salts thereof; and solvent system suitable for injection, wherein the solvent system comprises the mixture of water and ethanol.

The invention also relates to a stable liquid ready-to-use injectable formulation comprising bortezomib, or pharmaceutically acceptable salts thereof; solubilizer, and a solvent system.

The invention further relates to a stable liquid ready-to-use injectable formulation comprising bortezomib, or pharmaceutically acceptable salts thereof, solubilizer and a solvent system, wherein the solvent system comprises less than about 50% v/v of non-aqueous solvents selected from the group of ethanol, methanol, acetone, acetonitrile, 1-Butanol, 2-butanol, tertiary butanol, isopropanol, 1-propanol, dimethyl sulfoxide and di methylacetamide.

The invention further relates to a stable liquid ready-to-use injectable formulation comprising bortezomib, or pharmaceutically acceptable salts thereof, solubilizer and a solvent system, wherein the solvent system comprises non-aqueous solvents mixture of aqueous and non-aqueous solvents.

As used herein, the term “solubilizer” refers to any substance which enhances the solubility of the drug in the solvents.

Suitable solubilizer comprises, but not limited to, carboxylic acid, sugar and tromethamine.

Suitable carboxylic acid comprises, but not limited to, citric acid, malic acid, tartaric acid, succinic acid, acetic acid and the like.

Suitable sugar comprises, but not limited to, monosaccharide, disaccharide and reduced sugars that are suitable for administration by subcutaneous and intravenous routes. Some examples of sugar includes, but not limited to, sucrose, fructose, trehalose, xylitol, mannitol, sorbitol and the like.

One embodiment of the invention relates to a stable liquid ready-to-use injectable formulation comprising bortezomib, or pharmaceutically acceptable salts thereof; solubilizer and a solvent system, wherein the solubilizer is present in an amount of about 0.01 mg/mL to about 50 mg/mL, preferably in an amount of 1 mg/mL to about 25 mg/mL. The quantity varies depending on the nature of the solubilizer used.

The invention further relates to a stable liquid ready-to-use pharmaceutical formulation comprising bortezomib, or pharmaceutically acceptable salts thereof; about 1 mg/mL to about 25 mg/mL of solubilizer selected from the group of carboxylic acid, sugar and tromethamine and a solvent system suitable for injection, wherein the solvent system comprises less than about 50% v/v of non-aqueous solvents selected from the group of ethanol, methanol, acetone, acetonitrile, 1-Butanol, 2-butanol, tertiary butanol, isopropanol, 1-propanol, dimethyl sulfoxide and dimethylacetamide.

The invention further relates to a stable liquid ready-to-use injectable formulation comprising bortezomib, or pharmaceutically acceptable salts thereof; about 5 mg/mL to about 20 mg/mL of solubilizer selected from the group of carboxylic acid, sugar and tromethamine and a solvent system suitable for injection, wherein the solvent system comprises mixture of aqueous and non-aqueous solvents selected from the group of ethanol, methanol, acetone, acetonitrile, 1-Butanol, 2-butanol, tertiary butanol, isopropanol, 1-propanol, dimethyl sulfoxide and dimethylacetamide.

The invention further relates to a stable liquid ready-to-use injectable formulation comprising bortezomib, or pharmaceutically acceptable salts thereof; about 5 mg/mL to about 20 mg/mL of citric acid and a solvent system suitable for injection, wherein the solvent system comprises mixture of water and ethanol.

The invention further relates to a stable liquid ready-to-use injectable formulation comprising bortezomib, or pharmaceutically acceptable salts thereof; about 5 mg/mL to about 20 mg/mL of citric acid and a solvent system suitable for injection, wherein the solvent system comprises mixture of water and ethanol and wherein solvent system comprises about 40% v/v to about 75% v/v of ethanol,

The invention further relates to a stable liquid ready-to-use injectable formulation comprising bortezomib, or pharmaceutically acceptable salts thereof; solubilizer, buffering agent and a solvent system.

The amount of the bortezomib that can be solubilized is dependent on several parameters. One such parameter is pH. Higher pH results in poorer solubility of a basic compound, and lower pH would be expected to decrease the solubility of an acidic compound, as is known in the art. However, a pH should be selected to provide suitable stability of the proteasome inhibitor. For the purpose of this invention, the pH of the stable liquid ready-to-use injectable formulation comprising bortezomib, or pharmaceutically acceptable salts thereof will vary from about 2.5 to about 8. More preferably, it varies from about pH 3 to about 6. Standard modifications of the formulation can provide formulations of various pH within the contemplation of this invention.

A primary source of pH control can be buffer. Typically, a buffer is present as an acid or a base and its conjugate base or acid, respectively. According to the present disclosure the buffering agent may comprise at least one buffering agent selected from the group consisting of citrate (sodium or potassium), acetate, phosphate, malate, lactate, Tromethamine (Tris) and mixtures thereof. The most preferably used buffering agent is sodium citrate. In certain embodiments the buffering agent may be present in an amount of about 0.01 mg/mL to about 50 mg/mL, more preferably from about 1 mg/mL to about 45 mg/mL and most preferably from about 5 mg/mL to about 40 mg/mL. The amount of buffering agent used differs based on the buffering agent used in the composition.

Another embodiment of the invention relates to a stable liquid ready-to-use injectable composition comprising bortezomib, or pharmaceutically acceptable salts thereof; solubilizer, buffering agent and a solvent system suitable for injection, wherein the solvent system comprises the mixture of aqueous and non-aqueous solvents and wherein the solvent system comprises less than about 50% v/v of non-aqueous solvents selected from the group of ethanol, methanol, acetone, acetonitrile, 1-Butanol, 2-butanol, tertiary butanol, isopropanol, 1-propanol, dimethyl sulfoxide and di methylacetamide.

A further embodiment of the invention relates to a stable liquid ready-to-use injectable composition comprising bortezomib, or pharmaceutically acceptable salts thereof; of solubilizer selected from the group of carboxylic acid, sugar and tromethamine, buffering agent and a solvent system suitable for injection, wherein the solvent system comprises the mixture of aqueous and non-aqueous solvents and wherein the solvent system comprises from about 5% v/v to about 15% v/v of non-aqueous solvents selected from the group of ethanol, methanol, acetone, acetonitrile, 1-Butanol, 2-butanol, tertiary butanol, isopropanol, 1-propanol, dimethyl sulfoxide and di methylacetamide.

Another embodiment of the invention relates to a stable liquid ready-to-use injectable composition comprising bortezomib, or pharmaceutically acceptable salts thereof; about 0.01 mg/mL to about 50 mg/mL of solubilizer selected from the group of carboxylic acid, sugar and tromethamine, about 1 mg/mL to about 45 mg/mL of buffering agent and a solvent system suitable for injection, wherein the solvent system comprises the mixture of aqueous and non-aqueous solvents and wherein the solvent system comprises from about 5% v/v to about 15% v/v of non-aqueous solvents selected from the group of ethanol, methanol, acetone, acetonitrile, 1-Butanol, 2-butanol, tertiary butanol, isopropanol, 1-propanol, dimethyl sulfoxide and dimethylacetamide.

A further embodiment of the invention relates to a stable liquid ready-to-use injectable composition comprising bortezomib, or pharmaceutically acceptable salts thereof; about 0.01 mg/mL to about 50 mg/mL of solubilizer, about 1 mg/mL to about 45 mg/mL of buffering agent and a solvent system suitable for injection, wherein the solvent system comprises the mixture of aqueous and non-aqueous solvents and wherein the solvent system comprises from about 5% v/v to about 15% v/v of non-aqueous solvents, and wherein the composition has a pH of about 3 to about 7.

Another embodiment of the invention relates to a stable liquid ready-to-use injectable composition comprising bortezomib, or pharmaceutically acceptable salts thereof; citric acid, sodium citrate and a solvent system.

A further embodiment of the invention relates to a stable liquid ready-to-use injectable composition comprising bortezomib, or pharmaceutically acceptable salts thereof; citric acid, sodium citrate and a solvent system suitable for injection, wherein the solvent system comprises the mixture of water and ethanol and wherein the solvent system comprises less than about 50% v/v of ethanol.

Another embodiment of the invention relates to a stable liquid ready-to-use injectable composition comprising bortezomib, or pharmaceutically acceptable salts thereof; citric acid, sodium citrate and a solvent system suitable for injection, wherein the solvent system comprises the mixture of water and ethanol and wherein the solvent system comprises from about 5% v/v to about 15% v/v of ethanol.

One embodiment of the invention relates to a stable liquid ready-to-use injectable composition comprising bortezomib, or pharmaceutically acceptable salts thereof; about 0.01 mg/mL to about 50 mg/mL of citric acid, about 1 mg/mL to about 45 mg/mL of sodium citrate, and a solvent system suitable for injection, wherein the solvent system comprises the mixture of water and ethanol and wherein the solvent system comprises from about 5% v/v to about 15% v/v of ethanol.

Another embodiment of the invention relates to a stable liquid ready-to-use injectable composition comprising bortezomib, or pharmaceutically acceptable salts thereof; about 2.5 mg/mL of citric acid, about 6 mg/mL of sodium citrate and a solvent system suitable for injection, wherein the solvent system comprises the mixture of water and ethanol and wherein the solvent system comprises from about 5% v/v to about 15% v/v of ethanol.

A further embodiment of the invention relates to a stable liquid ready-to-use injectable composition comprising bortezomib, or pharmaceutically acceptable salts thereof; about 2.5 mg/mL of citric acid, about 6 mg/mL of sodium citrate and a solvent system suitable for injection, wherein the solvent system comprises the mixture of water and ethanol and wherein the solvent system comprises from about 5% v/v to about 15% v/v of ethanol and wherein the composition has a pH of about 3 to 7.

Another embodiment of the invention relates to a stable liquid ready-to-use injectable composition comprising bortezomib, or pharmaceutically acceptable salts thereof; solubilizer, buffering agent, stabilizer and a solvent system.

As used herein, the term “stabilizer” identifies an agent which improves the composition stability for a reasonable period of time, such as mentioned above, at certain temperatures. Suitable stabilizers include but are not limited to, sodium metabisulfite, sodium sulphite, sodium bisulfate, sodium thiosulfate, TPGS (d-alpha tocopheryl polyethylene glycol 1000 succinate), sodium acetate trihydrate, sodium formaldehyde sulfoxylate, tocopherol, ascorbic acid, EDTA and derivatives, monothioglycerol, methionine, thiourea, cysteine and propyl gallate. In one embodiment, preferred stabilizers are selected from the group of sodium metabisulfite, sodium formaldehyde sulfoxylate, tocopherol, ascorbic acid, EDTA and derivatives, monothioglycerol, and methionine.

Stabilizer is present in the amount of about 0.0001 mg/mL to about 10 mg/mL, more preferably in an amount of about 0.01 mg/mL to about 5 mg/mL and most preferably from about 0.1 mg/mL to about 4 mg/mL.

One embodiment of the invention relates to a stable liquid ready-to-use injectable composition comprising bortezomib, or pharmaceutically acceptable salts thereof; solubilizer selected from the group of carboxylic acid, sugar and tromethamine, buffering agent, stabilizers selected from the group of sodium metabisulfite, sodium formaldehyde sulfoxylate, tocopherol, ascorbic acid, EDTA and derivatives, monothioglycerol, and methionine and a solvent system suitable for injection, wherein the solvent system comprises the mixture of aqueous and non-aqueous solvents and wherein the solvent system comprises less than about 50% v/v of non-aqueous solvents selected from the group of ethanol, methanol, acetone, acetonitrile, 1-Butanol, 2-butanol, tertiary butanol, isopropanol, 1-propanol, dimethyl sulfoxide and di methylacetamide.

Another embodiment of the invention relates to a stable liquid ready-to-use injectable composition comprising bortezomib, or pharmaceutically acceptable salts thereof; solubilizer selected from the group of carboxylic acid, sugar and tromethamine, buffering agent, stabilizers selected from the group of sodium metabisulfite, sodium formaldehyde sulfoxylate, tocopherol, ascorbic acid, EDTA and derivatives, monothioglycerol, and methionine and a solvent system suitable for injection, wherein the solvent system comprises the mixture of aqueous and non-aqueous solvents and wherein the solvent system comprises from about 5% v/v to about 15% v/v of non-aqueous solvents selected from the group of ethanol, methanol, acetone, acetonitrile, 1-Butanol, 2-butanol, tertiary butanol, isopropanol, 1-propanol, dimethyl sulfoxide and di methylacetamide.

A further embodiment of the invention relates to a stable liquid ready-to-use injectable composition comprising bortezomib, or pharmaceutically acceptable salts thereof; about 0.01 mg/mL to about 50 mg/mL of solubilizer, about 1 mg/mL to about 45 mg/mL of buffering agent, from about 0.1 mg/mL to about 4 mg/ml of stabilizer and a solvent system suitable for injection, wherein the solvent system comprises the mixture of aqueous and non-aqueous solvents and wherein the solvent system comprises from about 5% v/v to about 15% v/v of non-aqueous solvents.

Another embodiment of the invention relates to a stable liquid ready-to-use injectable composition comprising bortezomib, or pharmaceutically acceptable salts thereof; about 0.01 mg/mL to about 50 mg/mL of solubilizer, about 1 mg/mL to about 45 mg/mL of buffering agent, from about 0.1 mg/mL to about 4 mg/ml of stabilizer and a solvent system suitable for injection, wherein the solvent system comprises the mixture of aqueous and non-aqueous solvents and wherein the solvent system comprises from about 5% v/v to about 15% v/v of non-aqueous solvents, and wherein the composition is from about wherein the composition has a pH of about 3 to about 7.

One embodiment of the invention relates to stable liquid ready-to-use injectable composition comprising bortezomib, or pharmaceutically acceptable salts thereof; citric acid, sodium citrate, EDTA and a solvent system.

Another embodiment of the invention relates to a stable liquid ready-to-use injectable composition comprising bortezomib, or pharmaceutically acceptable salts thereof; citric acid, sodium citrate, EDTA and a solvent system suitable for injection, wherein the solvent system comprises the mixture of water and ethanol and wherein the solvent system comprises less than about 50% v/v of ethanol.

A further embodiment of the invention relates to a stable liquid ready-to-use injectable composition comprising bortezomib, or pharmaceutically acceptable salts thereof; citric acid, sodium citrate, EDTA and a solvent system suitable for injection, wherein the solvent system comprises the mixture of water and ethanol and wherein the solvent system comprises from about 5% v/v to about 15% v/v of ethanol.

Another embodiment of the invention relates to a stable liquid ready-to-use injectable composition comprising bortezomib, or pharmaceutically acceptable salts thereof; about 0.01 mg/mL to about 50 mg/mL of citric acid, about 1 mg/mL to about 45 mg/mL of sodium citrate, about 0.5 mg/mL of EDTA and a solvent system suitable for injection, wherein the solvent system comprises the mixture of water and ethanol and wherein the solvent system comprises from about 5% v/v to about 15% v/v of ethanol.

A further embodiment of the invention relates to a stable liquid ready-to-use injectable composition comprising bortezomib, or pharmaceutically acceptable salts thereof; about 2.5 mg/mL of citric acid, about 6 mg/mL of sodium citrate and about 0.5 mg/mL of EDTA and a solvent system suitable for injection, wherein the solvent system comprises the mixture of water and ethanol and wherein the solvent system comprises from about 5% v/v to about 15% v/v of ethanol.

Another embodiment of the invention relates to a stable liquid ready-to-use injectable composition comprising bortezomib, or pharmaceutically acceptable salts thereof; about 2.5 mg/mL of citric acid, about 6 mg/mL of sodium citrate and about 0.5 mg/mL of EDTA and a solvent system suitable for injection, wherein the solvent system comprises the mixture of water and ethanol and wherein the solvent system comprises from about 5% v/v to about 15% v/v of ethanol and wherein the composition has a pH of about 3 to 7.

The stable liquid injectable pharmaceutical formulations of present invention may optionally include one or more pharmaceutically acceptable excipients. The pharmaceutically acceptable excipients may include any one or more of: one or more antibacterial preservatives, including one or more of phenyl mercuric nitrate, thiomersal, benzalkonium chloride, benzethonium chloride, phenol, cresol and chloro butanol; stabilizers including one or more of ascorbic acid, butylated hydroxy toluene (BHT), butylated hydroxy anisole (BHA), methionine, Pentetic acid, sodium sulfite, sodium bisulfite, Tocopherol, monothioglycerol, thymol, sodium formaldehyde sulfoxylate, propyl gallate, sodium ascorbate, sodium thiosulfate, sulfur dioxide, Vitamin E Polyethylene Glycol Succinate, potassium metabisulfite and sodium metabisulfite; and tonicity contributors including one or more of sodium chloride, potassium chloride, and alkaline substances including one or more of sodium hydroxide, potassium hydroxide, sodium carbonate and meglumine and salts such as sodium chloride.

The injectable formulations of the present invention have sufficient stability to have utility as a pharmaceutical agent. Preferably, the formulation has sufficient stability to allow its storage at a convenient temperature, preferably between 0° C. and 40° C., for a reasonable period of time.

The formulations of the present invention are particularly suited for use in parenteral administration. Injectable formulations may take any route including intramuscular, intra-peritoneal, intravenous or subcutaneous administration. Preferred are subcutaneous or intravenous route of administration.

Another embodiment of the present invention relates to the stable liquid ready-to-use injectable composition comprising bortezomib; wherein the injectable composition is packaged in a container suitable for both single and multi-use. The preferred containers include an ampoule, a vial, a pre-filled syringe, and intravenous bag. The preferred multi-use containers will contain bortezomib in an amount suitable to allow for at-least two distinct uses, more preferably for three, and most preferably for at least ten distinct uses (each of which may or may not require the same quantity of formulation administered to the patient). Thus, particularly preferred containers will be configured as a multi-use container (e.g., contain a volume of the composition that is suitable for multiple and independent administrations), and especially preferred multi-use containers include vials with a rubber stopper that can be pierced with a needle of a syringe.

The bortezomib formulations of the present invention can be packaged in any suitable sterile vial or container fit for the sterile storage of a pharmaceutical such as bortezomib for extended periods of time. Suitable containers can be glass vials, i.e. treated vials, molded glass vials, and CZ resin vials, polypropylene or polyethylene vials or other special purpose containers. Containers are of a size sufficient to hold one or more doses of bortezomib. The bortezomib formulations can also be stored in vials which are designed to minimize delamination and pitting problems, for example, PICVD (Plasma Impulse Chemical Vapor Deposition) and the like.

The stable multi-use injectable composition comprising bortezomib of the present invention will allow for the storage of the bortezomib for at least 1 week after first use, more preferably at least 2-4 weeks after first use, and most typically at least 1-3 months (and even longer) after first use without significant degradation of bortezomib under ambient conditions.

For the purpose of this invention stable liquid ready-to-use injectable formulation of bortezomib or pharmaceutically acceptable salts thereof comprise not more than about 10% of total impurities, expressed as percentages of the labeled bortezomib content.

In embodiments, the stable liquid ready-to-use injectable formulation of bortezomib or pharmaceutically acceptable salts thereof comprises not more than about 10% or 9.5% or 9% or 8.5% or 8% or 7.5% or 7% or 6.5% or 6% or 5.5% or 5% or 4.5% or 4% or 3.5% or 3% or 2.5% or 2% or 1.5% or 1% or 0.5% of total impurities.

For the purpose of this invention stable liquid ready-to-use injectable formulation of bortezomib or pharmaceutically acceptable salts thereof comprise not more than about 5% of single maximum individual impurity, expressed as percentages of the labeled bortezomib content.

In embodiments, the stable liquid ready-to-use injectable formulation of bortezomib or pharmaceutically acceptable salts thereof comprises not more than about 5% or 4.5% or 4% or 3.5% or 3% or 2.5% or 2% or 1.5% or 1% or 0.5% of single maximum individual impurity.

In embodiments, stable liquid ready-to-use injectable formulation of bortezomib or pharmaceutically acceptable salts thereof comprises not more than about 10% of total impurities and not more than about 5% of single maximum individual impurity.

In embodiments, stable liquid ready-to-use injectable formulation of bortezomib or pharmaceutically acceptable salts thereof comprises not more than about 8% of total impurities and not more than about 4% of single maximum individual impurity.

Details of impurities:

| Impurity | Chemical Name | Structure | RRT | RRF |
|---|---|---|---|---|
| Imp 2 | (S)-3-Phenyl-2-[(pyrazine-2- carbonyl)-amino]-propionic acid | 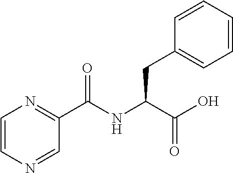 [SMILES: O=C(N[C@@H](Cc1ccccc1)C(=O)O)c1cnccn1] | 0.81 | 1.44 |
| Imp-6 | Pyrazine-2-carboxylic acid [1-(1- hydroxy-3-methyl- butylcarbamoyl)-2-phenyl- ethyl]-amide | 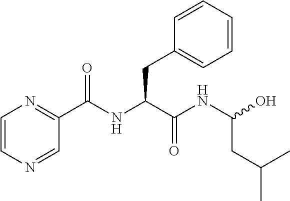 [SMILES: CC(C)CC(O)NC(=O)[C@H](Cc1ccccc1)NC(=O)c1cnccn1] | 1.54 | 0.95 |
| Imp-7 | Pyrazine-2-carboxylic acid [1-(1- hydroxy-3-methyl- butylcarbamoyl)-2-phenyl- ethyl]-amide. | 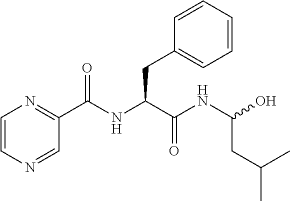 [SMILES: CC(C)CC(O)NC(=O)[C@H](Cc1ccccc1)NC(=O)c1cnccn1] | 1.80 | 0.96 |

The stable liquid ready-to-use injectable formulation of bortezomib or pharmaceutically acceptable salts thereof can be analyzed by common techniques, such as high performance liquid chromatography, to determine their drug content and the concentrations of drug-related impurities.

The pharmaceutical formulation of the present invention is resistant to color changes, degradation and ensures acceptable shelf life.

In another aspect of the invention provides methods of treating a bortezomib sensitive disease in mammals. Bortezomib sensitive diseases include, but are not limited to, cancers, such as multiple myeloma and mantle cell lymphoma. The methods include administering an effective amount of a bortezomib containing composition as described herein to a mammal in need thereof.

The following examples further describe certain specific embodiments of the invention and demonstrate the practice and advantages thereof. It is to be understood that the examples are given by way of illustration only and are not intended to limit the scope of the invention in any manner.

### EXAMPLES

### Examples 1-3

| | Examples | | |
|---|---|---|---|
| Ingredients | 1 | 2 | 3 |
| Bortezomib (mg/ml) | 2.5 | 2.5 | 2.5 |
| Citric acid (mg/ml) | 2.5 | 2.5 | 2.5 |
| Ethanol (ml) | 0.1 | 0.2 | 0.5 |
| Water (ml) | 0.9 | 0.8 | 0.5 |
| pH of the composition | 2.87 | 2.82 | 3.14 |

Manufacturing Process:

1. Required quantity of citric acid was taken and dissolved in ethanol.

2. Bortezomib was added to the solution of step 1.

3. Water was added to the solution of step 2 and purged with nitrogen.

4. The solution of bortezomib from step 3 was filtered through 0.22μ sterile filter.

5. Bortezomib solution of step 4 was filled into suitable vial and stoppered.

6. Vials prepared as per examples 1-3 were stored at 2-8° C., 25±2° C. and 60% relative humidity (RH). The contents of the initial and stored vials were analyzed for impurity content using suitable HPLC method and shown in Table 1.

TABLE 1

| Condition/ | Ex: | Impurities | | | |
|---|---|---|---|---|---|
| Time | No. | Total Imp | Imp-2 | Imp-6 | Imp-7 |
| Initial | 1 | 1.65 | 0.07 | 0.27 | 0.27 |
| | 2 | 1.22 | 0.28 | 0.17 | 0.27 |
| | 3 | 1.21 | 0.76 | 0.09 | 0.06 |
| 2-8° C. | 1 | 2.74 | 0.08 | 0.30 | 0.28 |
| 1 week | 2 | 0.87 | 0.17 | 0.13 | 0.13 |
| | 3 | 0.51 | 0.13 | 0.06 | 0.08 |
| 2-8° C. | 1 | 5.47 | 0.15 | 0.91 | 1.01 |
| 2 weeks | 2 | 6.04 | 0.52 | 1.23 | 0.16 |
| | 3 | 4.50 | 0.24 | 0.69 | 0.90 |
| 25° C./60% RH | 1 | 8.06 | 0.51 | 1.02 | 1.56 |
| 1 week | 2 | 1.45 | 1.19 | 0.05 | 0.09 |
| | 3 | 1.31 | 0.95 | 0.06 | 0.09 |
| 25° C./60% RH | 1 | 11.10 | 0.20 | 1.30 | 1.65 |
| 2 weeks | 2 | 11.50 | 2.95 | 2.11 | 3.40 |
| | 3 | 14.25 | 2.12 | 2.8 | 4.3 |

### Examples 4-6

| | Example | | |
|---|---|---|---|
| Ingredients | 4 | 5 | 6 |
| Bortezomib (mg/ml) | 2.5 | 2.5 | 2.5 |
| Citric acid (mg/ml) | 2.5 | 1.0 | 7.5 |
| Sodium citrate (mg/ml) | 6 | 2.4 | 18.0 |
| EDTA (mg/ml) | — | 0.5 | 0.5 |
| Ethanol (ml) | 0.1 | 0.1 | 0.5 |
| Water (ml) | 0.9 | 0.9 | 0.5 |
| pH of the composition | 5.17 | 5.06 | 5.39 |

Manufacturing Process:

1. Required quantity of citric acid was taken and dissolved in Ethanol.

2. Bortezomib was added to the solution of step 1.

3. Sodium citrate was dissolved in water and further EDTA was added in case of examples 5 and 6.

4. Bortezomib solution of in step 2 were added to the solution of step 3 and purged with nitrogen.

5. The solution of bortezomib of step 4 was filtered through 0.24 sterile filter.

6. Bortezomib solution of step 5 was filled into suitable vial, stoppered.

7. Vials prepared were stored at 2-8° C., 25±2° C. and 60% relative humidity (RH), or at 40±2° C. and 75% RH. The contents of the initial and stored vials are analyzed for impurity content using suitable HPLC method and shown in Table 2.

TABLE 2

| Condition/ | Ex: | Impurities | | | |
|---|---|---|---|---|---|
| Time | No. | Total Imp | Imp-2 | Imp-6 | Imp-7 |
| Initial | 4 | 0.80 | 0.06 | 0.43 | 0.17 |
| | 5 | 1.17 | 0.05 | 0.71 | 0.27 |
| | 6 | 0.90 | 0.09 | 0.14 | 0.11 |
| 2-8° C. | 4 | 4.40 | ND | 3.24 | 1.00 |
| 2 weeks | 5 | 7.63 | 0.14 | 5.00 | 1.90 |
| | 6 | 0.96 | 0.12 | 0.13 | 0.11 |
| 25° C./60% RH | 4 | 4.55 | 0.64 | 2.63 | 1.27 |
| 2 weeks | 5 | 5.88 | 0.40 | 3.66 | 1.67 |
| | 6 | 1.18 | 0.33 | 0.13 | 0.13 |
| 40° C./75% RH | 4 | 8.23 | 2.82 | 2.63 | 1.8 |
| 2 weeks | 5 | 7.0 | 2.8 | 2.9 | 1.1 |
| | 6 | 3.17 | 1.39 | 0.15 | 0.19 |

From data presented in table 1, it is evident that liquid formulation can be prepared using less than about 50% v/v of ethanol as non-aqueous solvent. Addition of buffering agent and/or stabilizer like EDTA further reduces degradation of bortezomib in such liquid composition which is evident form results as mentioned in Table 2. Moreover, it was found that higher amount of buffer imparts further stability into such liquid formulations.

### Example 7-10

| | Example | | | |
|---|---|---|---|---|
| Ingredients | 7 | 8 | 9 | 10 |
| Bortezomib | 2.5 | 2.5 | 2.5 | 2.5 |
| (mg/ml) | | | | |
| Citric acid | 7.5 | 7.5 | 7.5 | 7.5 |
| (mg/ml) | | | | |
| Sodium citrate | 18 | 40 | 18 | 40 |
| (mg/ml) | | | | |
| Sodium meta | 2 | 1.5 | 2 | 2 |
| bisulfite | | | | |
| (mg/ml) | | | | |
| Ethanol (ml) | 0.5 | 0.25 | 0.08* | 0.02 |
| Water (ml) | q.s. to 1 ml | q.s. to 1 ml | q.s. to 1 ml | q.s. to 1 ml |
| pH of the | 5.48 | 5.60 | 5.47 | 5.10 |
| composition | | | | |
| Osmolality | 8440 | 5500 | Approx. | — |
| (mOsm/kg) | | | 2581 | |

*0.25 ml of Ethanol was evaporated in process to reduce to 0.08 ml in example 9.

Manufacturing Process:

1. Required quantity of citric acid was taken and dissolved in Ethanol.

2. Bortezomib was added to the solution of step 1.

3. Sodium citrate was dissolved in water.

4. Bortezomib solution of in step 2 were added to the solution of step 3 and purged with nitrogen.

5. For example 9, excess ethanol was evaporated using nitrogen purging while maintaining bulk solution temperature at 25° C.

6. Sodium meta bisulfite was added in solution of step 4 (step 5 in case of example 9) and volume was made up with water.

7. The solution of bortezomib of step 6 was filtered through 0.22μ sterile filter.

8. Bortezomib solution of step 7 was filled into suitable vial, stoppered.

9. Vials prepared were stored at 2-8° C., 25±2° C. and 60% relative humidity (RH).

The contents of the initial and stored vials are analyzed for impurity content using suitable HPLC method and shown in Table 3.

TABLE 3

| Condition/ | Ex: | Impurities | | | |
|---|---|---|---|---|---|
| Time | No. | Total Imp | Imp-2 | Imp-6 | Imp-7 |
| Initial | 7 | 0.17 | 0.025 | ND | ND |
| | 8 | 0.32 | 0.029 | 0.104 | 0.068 |
| | 9 | 0.28 | 0.019 | 0.105 | 0.039 |
| | 10 | 0.49 | 0.032 | 0.259 | 0.062 |
| 2-8° C. | 7 | 0.20 | 0.050 | 0.051 | ND |
| 1 Month | 8 | 0.44 | 0.063 | 0.184 | 0.123 |
| | 9 | 0.34 | 0.060 | 0.098 | 0.078 |
| | 10 | 0.82 | 0.162 | 0.108 | 0.127 |
| 2-8° C. | 7 | 0.33 | 0.188 | 0.022 | ND |
| 3 Months | 8 | 0.58 | 0.019 | 0.191 | 0.095 |
| | 9 | 0.36 | 0.165 | 0.070 | 0.055 |
| | 10 | 1.29 | 0.374 | 0.225 | 0.335 |
| 25° C./60% RH | 7 | 0.78 | 0.441 | 0.041 | 0.043 |
| 1 Month | 8 | 0.85 | 0.31 | 0.20 | 0.15 |
| | 9 | 1.05 | 0.569 | 0.12 | 0.13 |
| | 10 | 3.19 | 1.527 | 0.55 | 0.81 |
| 25° C./60% RH | 7 | — | — | — | — |
| 3 Months | 8 | 2.31 | 1.44 | 0.13 | 0.20 |
| | 9 | 1.92 | 1.54 | 0.12 | 0.09 |
| | 10 | 5.31 | 3.90 | 0.26 | 0.25 |

From data presented in table 3, it is evident that all liquid formulations comprising less than about 50% v/v of ethanol as non-aqueous solvent are stable for commercially relevant time.

Local Tolerance Study of Formulations of Example 7-9:

A single dose local tolerance study was performed in rabbits by administering formulation of Example 7 to 9 by subcutaneous route at Lateral Thorax and compared with respective placebo and Saline in same animals. Erythema & Eschar and Oedema at injection site were observed and graded according to pre-determined scale. Such scores for each test formulation with respective placebo and Saline are captured in Table 4.

TABLE 4

| | | Time in Hrs post application | | | | | | | | | | | | | |
|---|---|---|---|---|---|---|---|---|---|---|---|---|---|---|---|
| | | 0.25 | 0.5 | 1 | 2 | 3 | 4 | 6 | 12 | 24 | 48 | 72 | 96 | | |
| Skin Reactions | | Grading of skin reactions | | | | | | | | | | | | | |
| Ex. 7 | Erythema | 0 | 0 | 0 | 0 | 0 | 0 | 0 | 0 | 0 | 2 | 2 | 2 | Animal | |
| | & Eschar | | | | | | | | | | | | | found dead | |
| | Oedema | 0 | 0 | 1 | 2 | 1 | 1 | 1 | 1 | 1 | 2 | 2 | 2 | at morning | |
| Placebo | Erythema | 0 | 0 | 0 | 0 | 0 | 0 | 0 | 0 | 0 | 0 | 0 | 0 | observation | |
| | & Eschar | | | | | | | | | | | | | of Day 6 | |
| | Oedema | 0 | 0 | 0 | 2 | 2 | 1 | 1 | 1 | 1 | 1 | 0 | 0 | | |
| Normal | Erythema | 0 | 0 | 0 | 0 | 0 | 0 | 0 | 0 | 0 | 0 | 0 | 0 | | |
| Saline | & Eschar | | | | | | | | | | | | | | |
| | Oedema | 0 | 0 | 0 | 0 | 0 | 0 | 0 | 0 | 0 | 0 | 0 | 0 | | |
| Ex.8 | Erythema | 0 | 0 | 0 | 0 | 0 | 0 | 0 | 0 | 0 | 2 | 2 | 1 | Animal | |
| | & Eschar | | | | | | | | | | | | | found dead | |
| | Oedema | 0 | 0 | 0 | 1 | 1 | 1 | 1 | 1 | 1 | 1 | 1 | 1 | after | |
| Placebo | Erythema | 0 | 0 | 0 | 0 | 0 | 0 | 0 | 0 | 0 | 0 | 0 | 0 | morning | |
| | & Eschar | | | | | | | | | | | | | observation | |
| | Oedema | 0 | 0 | 0 | 0 | 0 | 0 | 0 | 0 | 0 | 0 | 0 | 0 | of Day 5 | |
| Normal | Erythema | 0 | 0 | 0 | 0 | 0 | 0 | 0 | 0 | 0 | 0 | 0 | 0 | (~100 hrs | |
| Saline | & Eschar | | | | | | | | | | | | | post dose) | |
| | Oedema | 0 | 0 | 0 | 0 | 0 | 0 | 0 | 0 | 0 | 0 | 0 | 0 | | |
| | | Time in Hrs post application | | | | | | | | | | | | | |
| | | 0.25 | 0.5 | 1 | 2 | 3 | 4 | 6 | 12 | 24 | 48 | 72 | 96 | 120 | 168 |
| Skin Reactions | | Grading of skin reactions | | | | | | | | | | | | | |
| Ex. 9 | Erythema | 0 | 0 | 0 | 0 | 0 | 0 | 0 | 0 | 1 | 1 | 0 | 0 | 0 | 0 |
| | & Eschar | | | | | | | | | | | | | | |
| | Oedema | 0 | 0 | 0 | 0 | 0 | 0 | 0 | 0 | 1 | 1 | 0 | 0 | 0 | 0 |
| Placebo | Erythema | 0 | 0 | 0 | 0 | 0 | 0 | 0 | 0 | 0 | 0 | 0 | 0 | 0 | 0 |
| | & Eschar | | | | | | | | | | | | | | |
| | Oedema | 0 | 0 | 0 | 0 | 0 | 0 | 0 | 0 | 0 | 0 | 0 | 0 | 0 | 0 |
| Normal | Erythema | 0 | 0 | 0 | 0 | 0 | 0 | 0 | 0 | 0 | 0 | 0 | 0 | 0 | 0 |
| Saline | & Eschar | | | | | | | | | | | | | | |
| | Oedema | 0 | 0 | 0 | 0 | 0 | 0 | 0 | 0 | 0 | 0 | 0 | 0 | 0 | 0 |

From the rabbit study it was observed that formulation of Example 7 to 9 has local irritation potential compared to saline, however, such local irritation was significantly reduced by lowering the concentration of ethanol i.e. non-aqueous solvent.

Hence, stable liquid ready-to-use formulation of this invention was found to be suitable for injection without compromising impurity profile as well as irritation potential.

1. A stable liquid ready-to-use injectable formulation of bortezomib or pharmaceutically acceptable salts thereof comprising solvent system suitable for injection, wherein the solvent system comprises less than about 50% v/v of non-aqueous solvent.

2. The stable liquid ready-to-use injectable formulation of claim 1, wherein the formulation comprises from about 0.1 mg/ml to about 15 mg/ml of bortezomib or pharmaceutically acceptable salts thereof.

3. The stable liquid ready-to-use injectable formulation of claim 2, wherein the formulation comprises from about 0.5 mg/ml to about 5 mg/ml of bortezomib or pharmaceutically acceptable salts thereof.

4. The stable liquid ready-to-use injectable formulation of claim 1, wherein the solvent system comprises less than about 30% v/v of non-aqueous solvent.

5. The stable liquid ready-to-use injectable formulation of claim 4, wherein the solvent system comprises less than about 20% v/v of non-aqueous solvent.

6. The stable liquid ready-to-use injectable formulation of claim 5, wherein the solvent system comprises from about 0% v/v to about 15% w/v of non-aqueous solvent.

7. The stable liquid ready-to-use injectable formulation of claim 1, wherein the non-aqueous solvent is selected from the group of ethanol, methanol, acetone, acetonitrile, 1-Butanol, 2-butanol, tertiary butanol, isopropanol, 1-propanol, dimethyl sulfoxide and dimethylacetamide.

8. The stable liquid ready-to-use injectable formulation of claim 7, wherein the non-aqueous solvent is ethanol.

9. The stable liquid ready-to-use injectable formulation of claim 7, wherein the non-aqueous solvent is acetonitrile.

10. The stable liquid ready-to-use injectable formulation of claim 7, wherein the non-aqueous solvent is tertiary butanol.

11. The stable liquid ready-to-use injectable formulation of claim 1, wherein the formulation further comprises at least one pharmaceutically acceptable excipients selected form the group of solubilizer, stabilizer, buffering agent, tonicity contributing agent, pH adjusting agent.

12. The stable liquid ready-to-use injectable formulation of claim 11, wherein the solubilizer is selected form the group of carboxylic acid or derivative thereof, tromethamine and sugar.

13. The stable liquid ready-to-use injectable formulation of claim 12, wherein the solubilizer is citric acid or derivative thereof, tromethamine or mannitol.

14. The stable liquid ready-to-use injectable formulation of claim 12, comprising solubilizer in an amount from about 0.01 mg/mL to about 50 mg/mL.

15. The stable liquid ready-to-use injectable formulation of claim 11, wherein the stabilizer is selected form the group of sodium metabisulfite, sodium sulphite, sodium bisulfate, sodium thiosulfate, TPGS (d-alpha tocopheryl polyethylene glycol 1000 succinate), sodium acetate trihydrate, sodium formaldehyde sulfoxylate, tocopherol, ascorbic acid, EDTA and derivatives, monothioglycerol, methionine, thiourea, cysteine, butylated hydroxy toluene (BHT), butylated hydroxy anisole (BHA) and propyl gallate.

16. The stable liquid ready-to-use injectable formulation of claim 15, wherein the stabilizer is selected form the group of sodium metabisulfite, sodium formaldehyde sulfoxylate, tocopherol, ascorbic acid, EDTA and derivatives, monothioglycerol, and methionine.

17. The stable liquid ready-to-use injectable formulation of claim 15, comprising stabilizer in an amount from about from about 0.01 mg/mL to about 10 mg/mL.

18. The stable liquid ready-to-use injectable formulation of claim 11, wherein the buffering agent is selected form the group of sodium acetate, acetic acid, sodium citrate, citric acid, tromethamine and phosphate.

19. The stable liquid ready-to-use injectable formulation of claim 1, wherein the formulation is having a pH ranging from about 2.5 to about 8.

20. The stable liquid ready-to-use injectable formulation of claim 19, wherein the formulation is having a pH ranging from about 3 to about 6.

21. The stable liquid ready-to-use injectable formulation of claim 1, wherein the formulation comprises not more than about 10% of total impurities.

22. The stable liquid ready-to-use injectable formulation of claim 21, wherein the formulation comprises not more than about 10% of total impurities and not more than about 5% of single maximum individual impurity.

23. The stable liquid ready-to-use injectable formulation of claim 22, wherein the formulation comprises not more than about 8% of total impurities and not more than about 4% of single maximum individual impurity.
